(19) United States
(12) Patent Application Publication
(10) Pub. No.: US 2022/0160620 A1
(43) Pub. Date: May 26, 2022

(54) FORMULATIONS FOR TREATING PSORIASIS

(71) Applicant: Galia SALEH, Arncliffe, NSW (AU)

(72) Inventor: Galia SALEH, Arncliffe, NSW (AU)

(21) Appl. No.: 17/601,463

(22) PCT Filed: Apr. 3, 2020
(86) PCT No.: PCT/AU2020/050340

§ 371 Date: Oct. 5, 2021

(30) Foreign Application Priority Data
Apr. 5, 2019 (AU) 2019901180

(51) Int. Cl. A61K 9/00; A61K 36/8998; A61K 36/889; A61K 47/44; A61K 9/06; A61K 45/06

(52) U.S. Cl. CPC A61K 9/0014; A61K 36/8998; A61K 45/06; A61K 47/44; A61K 9/06; A61K 36/889

(57) ABSTRACT

Disclosed herein is a topical formulation for treating psoriasis. The formulation comprises ground wheat and a wheat extract, ground barley and a barley extract, an oil and egg or a fraction thereof. Also disclosed is a method for treating psoriasis comprising applying the topical formulation to an affected area of a patient.

### TECHNICAL FIELD

The present invention relates to formulations and methods for treating psoriasis.

### BACKGROUND ART

Psoriasis is an autoimmune condition that reportedly affects up to 4% of the population. Psoriasis may present in a number of forms, and is generally characterized by patches of abnormal skin which are typically red, dry, itchy and scaly. Psoriasis can vary in severity, ranging from small, localized patches to complete body coverage.

There is no cure for psoriasis, although some treatments are available that can help to control the symptoms. Such treatments include steroid-containing creams, vitamin D3 cream and immune system suppressing medications. However, some of these treatments carry a risk of side effects and many are not indicated for children. Indeed, the majority of the non-topical treatments for psoriasis are not indicated for children, which significantly limits treatment options for children suffering from psoriasis. Furthermore, most topical formulations for treating psoriasis contain steroids, which can produce unacceptable side effects in children (and others), and which may lead to a reluctance by parents to use such formulations on their children.

It would be advantageous to provide treatments for psoriasis that provide effective relief from symptoms but without the risk of adverse side effects associated with systemically delivered or steroid-containing treatments. Treatments that are perceived as being more “Natural” may also result in improved patient compliance.

### SUMMARY OF INVENTION

In a first aspect, the present invention provides a topical formulation for treating psoriasis. The formulation comprises ground wheat and a wheat extract, ground barley and a barley extract, an oil and egg or a fraction thereof.

The formulation of the present invention moisturises and hydrates the patient's epidermis by creating a protective barrier which slows down and maintains the usual hallmark of rapid regeneration of skin present within psoriasis. The formulation assists in treating the psoriasis by keeping the skin and scalp smooth, soft, and non-flaky by reducing scaling, dryness, irritation, and inflammation within the skin.

The formulation of the present invention is an improvement over existing treatment regimens because its ingredients do not include steroids or other substances associated with a significant risk of adverse side effects (e.g. sun sensitivity, in the case of steroidal treatments). The formulation contains ingredients that are generally regarded as safe and/or as being “Natural”, thus providing patients suffering with psoriasis (and their parents, in the case of children) ease of mind about what they are using on their bodies.

In some embodiments, the ground wheat may comprise wheat flour. In some embodiments, the ground wheat may comprises ground Triticum aestivum and/or Triticum vulgare, two strains of common wheat. Similarly, in some embodiments, the wheat extract may comprise Triticum aestivum germ extract and/or Triticum vulgare germ extract.

In some embodiments, the ground barley may be obtained from one or more of the following: pearl barley, malted barley and hulled barley. In some embodiments, the strain of barley used may be Hordeum vulgare, with ground Hordeum vulgare and/or Hordeum vulgare extract being included in the formulation.

In some embodiments, the oil may comprise olive oil and/or Cocos nucifera oil (also known as coconut oil).

In some embodiments, the egg or a fraction thereof may be provided in the form of whole egg, egg albumen, egg vitellus or extracted therapeutically active (i.e. in the context of treating psoriasis) fractions of the egg. The egg or a fraction thereof may be provided in the form of combinations of these components.

In some embodiments, the formulation may further comprise a fatty acid. Such a fatty acid may, for example, be selected from one or more of the following: oleic acid, linoleic acid and palmitic acid.

In some embodiments, the formulation may further comprise a fatty alcohol. Such a fatty alcohol may, for example, be cetyl alcohol.

In some embodiments, the formulation may further comprise Vitamin E or a component thereof (e.g. gamma-tocopherol and/or alpha-tocopherol).

In a second aspect, the present invention provides a topical formulation for treating psoriasis. The formulation comprises:
ground Triticum aestivum and ground Triticum vulgare;
Triticum aestivum germ extract and Triticum vulgare germ extract;
ground Hordeum vulgare;
Hordeum vulgare extract;
Cocos nucifera oil;
egg albumen;
egg vitellus;
oleic acid;
linoleic acid;
palmitic acid;
cetyl alcohol;
Cocos nucifera water;
Vitamin E;
gamma-tocopherol; and
alpha-tocopherol.

In a third aspect, the present invention provides a method for treating psoriasis. The method comprises applying the topical formulation of the first or second aspect of the present invention to an affected area of a patient.

In some embodiments, the applied topical formulation may be allowed to remain on the patient for an extended period of time (e.g. overnight).

In some embodiments, the topical formulation is applied on a daily basis until treatment is complete, which may, for example, take between 1-5 days.

In some embodiments, the topical formulation may be re-applied on a regular basis (e.g. weekly or monthly) in order for the patient to remain substantially psoriasis free.

In a fourth aspect, the present invention provides the use of topical formulation of the first or second aspect of the present invention for treating psoriasis

The inventor notes that the topical formulations of the first and second aspects of the present invention may also be used to treat skin conditions other than psoriasis. The inventor has, for example, successfully treated a patient with relatively severe eczema and expects that it may be used to treat other skin conditions. In other aspects, therefore, the present invention provides a formulation having the composition described above, which is suitable for treating eczema and other skin conditions. The present invention also provides methods for treating eczema and other skin conditions that have the same steps as the method of the third aspect.

Additional features and advantages of the various aspects of the present invention will be described below in the context of specific embodiments. It will be appreciated, however, that such additional features may have a more general applicability in the present invention than that described in the context of these specific embodiments.

### DESCRIPTION OF EMBODIMENTS

The overarching purpose of the present invention is to provide a formulation which is effective in the treatment of psoriasis, but which does not contain ingredients that might cause adverse side effects (especially in children).

The present invention thus provides a topical formulation for treating psoriasis. The formulation comprises ground wheat and a wheat extract, ground barley and a barley extract, an oil (or mixture of oils) and egg or a fraction thereof. The functionality of each of these components, and their unexpected effects in combination, will be described in further detail below.

The formulations of the present invention are envisaged to be useful for the treatment of psoriasis, based on the promising observations and patient trials described in further detail below. In summary, the inventor has observed that, when topically applied to the skin of a patient exhibiting symptoms of psoriasis, formulations in accordance with the present invention have relieved the inflammation, scaling, itchiness and irritation of the psoriasis. As noted above, the inventor has successfully treated a patient with relatively severe eczema and expects that the formulation will also be useful more generally as a treatment of other skin conditions, due to it relieving redness, dryness, scaling, and itchiness whilst hydrating the skin.

The ingredients present in the formulation assist by moisturising and hydrating the epidermis by creating a protective barrier, which the inventor has found slows down and maintains the usual hallmark of rapid regeneration of skin present within psoriasis. The formulation assists treatment by keeping the skin and scalp smooth, soft, and non-flaky by reducing scaling, dryness, irritation, and inflammation within the skin.

The formulation of the present invention provides an improvement over existing formulations as it is highly safe to use, is non-toxic and steroid free, which helps give patients suffering from psoriasis ease of mind about what they are using on their bodies. It is highly safe and can be used on children of all ages. As noted above, many current psoriasis treatments are not child friendly, therefore limiting treatment options for children suffering from psoriasis.

The formulations of the present invention include ground wheat and a wheat extract, which provides a skin nourishing function. Any suitable strain of wheat may be used in the formulation, with the common wheat strains of Triticum aestivum and Triticum vulgare being readily available and relatively cheap.

The ground wheat may be provided in any suitable form, provided that its beneficial effects are imparted to the formulation. In some embodiments, for example, the ground wheat may comprise or consist of wheat flour, preferably wholemeal flour. Alternatively, a more coarsely ground wheat product may be used, for example if the topical formulation requires a degree of coarseness.

Similarly, the wheat extract may be provided in any suitable form. The wheat extract may, for example, comprise or consist of Triticum aestivum germ extract, which imparts skin nourishing and anti-aging effects. The wheat extract may, for example, comprise or consist of Triticum vulgare germ extract, which imparts skin conditioning effects. Such extracts are commercially available, or can be easily produced, for example, by soaking the wheat overnight in water and using the water as the wheat extract.

The formulation may include any effective amount of the ground wheat and wheat extract, and it is within the ability of a person skilled in the art to ascertain such an effective amount, using no more that routine trials and experimentation and based on factors such as the ingredients in the formulation, the consistency the formulation requires and the severity of the condition being treated. In some embodiments, for example, the formulation may contain between about 2 wt % and 10 wt % (e. g. between about 3 wt % and 9 wt %, between about 4 wt % and 8 wt % or between about 5 wt % and 7 wt %) of each of the ground wheat and wheat extract. In some embodiments, for example, the formulation may contain about 2 wt %, 3 wt %, 4 wt %, 5 wt %, 6 wt %, 7 wt %, 8 wt %, 9 wt % or 10 wt % of each of the ground wheat and wheat extract.

The formulations of the present invention also include ground barley and a barley extract, which, can help to increase skin elasticity.

The ground barley may be provided in any suitable form, provided that its beneficial effects are imparted to the formulation. In some embodiments, for example, the ground barley may be obtained by grinding pearly barley, malted barley and/or hulled barley. Any suitable strain of barley may be used in the formulation, with the common barley strains of Hordeum vulgare and Hordeum vulgare being readily available and relatively cheap.

Similarly, the barley extract may be provided in any suitable form. One particular barley extract the inventor has found to be effective and readily commercially available is an extract of roasted malted barley.

The formulation may include any effective amount of the ground barley and barley extract, and it is within the ability of a person skilled in the art to ascertain such an effective amount, using no more that routine trials and experimentation and based on factors such as the ingredients in the formulation, the consistency the formulation requires and the severity of the condition being treated. In some embodiments, for example, the formulation may contain between about 2 wt % and 10 wt % (e. g. between about 3 wt % and 9 wt %, between about 4 wt % and 8 wt % or between about 5 wt % and 7 wt %) of each of the ground barley and barley extract. In some embodiments, for example, the formulation may contain about 2 wt %, 3 wt %, 4 wt %, 5 wt %, 6 wt %, 7 wt %, 8 wt %, 9 wt % or 10 wt % of each of the ground barley and barley extract.

The formulations of the present invention also include an oil which, in addition to acting as a carrier for other components and imparting an appropriate consistency to the formulation, assists in moisturising the skin and preventing aging and cell damage. Any oil may be used in the formulation, provided that it is compatible with the other ingredients and has physical characteristics that are consistent with a formulation that is to be topically applied to a patient's skin.

Suitable oils trialled by the inventor, for example, include olive oil and Cocos nucifera oil (also known as coconut oil). Coconut oil contains fatty acids, vitamins and antioxidants, and has beneficial conditioning and moisturising properties. In some embodiments, it may be beneficial for the formulation to contain combinations of oils.

The formulation may include any effective amount of the oil(s), and it is within the ability of a person skilled in the art to ascertain such an effective amount using no more that routine trials and experimentation and based on factors such as the ingredients in the formulation, the consistency the formulation requires and the severity of the condition being treated. In some embodiments, for example, the formulation may contain between about 10 wt % and 30 wt % (e. g. between about 10 wt % and 25 wt %, between about 10 wt % and 20 wt % or between about 15 wt % and 20 wt %) of the oil(s). In some embodiments, for example, the formulation may contain about 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, or 30 wt % of the oil(s).

Finally, the formulations of the present invention also include egg or a fraction thereof, which helps to emulsify the ingredients in the formulation and also moisturises and improves skin texture.

The egg or a fraction thereof may be provided to the formulation in any therapeutically effective (i.e. which, in combination with the other components of the formulation treats psoriasis) form. The egg or a fraction thereof may, for example, be provided in the form of whole egg, egg albumen, egg vitellus or extracted therapeutically active fractions of the egg.

The formulation may include any effective amount of the egg or egg fraction, and it is within the ability of a person skilled in the art to ascertain such an effective amount using no more that routine trials and experimentation and based on factors such as the ingredients in the formulation, the consistency the formulation requires and the severity of the condition being treated. In some embodiments, for example, the formulation may contain between about 5 wt % and 30 wt % (e. g. between about 5 wt % and 25 wt %, between about 10 wt % and 20 wt % or between about 10 wt % and 15 wt %) of the egg or egg fraction. In some embodiments, for example, the formulation may contain about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, or 30 wt % of the egg or egg fraction.

In specific embodiments of the present invention, the formulation may comprise the ingredients shown in Table 1, below.

TABLE 1
Composition of Formulation 1

| | Effect contributed to | |
|---|---|---|
| Ingredient | the formulation | Preferred amount (wt %) |
| Wheat | Assists by nourishing | between about 2 and 10 wt % |
| | the skin. | |
| Wheat | Assists by nourishing | between about 2 and 10 wt % |
| extract | the skin. | |
| Barley | Increases the | between about 2 and 10 wt % |
| | elasticity of the skin. | |
| Barley | Increases the | between about 2 and 10 wt % |
| extract | elasticity of the skin. | |
| Olive oil | Highly moisturising | between about 10 and 30 wt % |
| | for the skin and | |
| | prevents the body | |
| | from aging and cell | |
| | damage. | |
| Egg | Emulsifies the | between about 5 and 30 wt % |
| | ingredients to | |
| | combine. | |
| | Moisturises skin and | |
| | improves skin texture. | |

The formulation may also contain water. The water may simply be distilled water, or may be a water obtained from natural sources, such as Cocos nucifera water (more commonly referred to as coconut water). Coconut water may have antioxidant, moisturizing, revitalizing and anti-aging properties.

The topical formulation of the present invention may also include additional ingredients, where such further ingredients provide beneficial effects and do not deleteriously affect the formulation's activity.

In some embodiments, for example, the formulation may further comprise a fatty acid in order to impart desirable physical characteristic (e.g. consistency) to the resultant formulation and also because of the beneficial properties to the skin of many fatty acids.

Any fatty acid that is compatible with other components of the formulation (i.e. will not react with other components or potentially cause stability issues) and capable of being topically applied may be used. Fatty acids specifically trialled by the inventor include oleic acid, linoleic acid and palmitic acid, which should impart anti-inflammatory properties on the resultant formulation. Linoleic acid, for example, is used in the beauty products industry because of its beneficial properties (e.g. anti-inflammatory, acne reductive, skin-lightening and moisture retentive properties) on the skin. Similarly, palmitic acid is routinely used to produce a soaps and cosmetics. Combinations of two or more fatty acids may be used, where appropriate.

In some embodiments, for example, the formulation may further comprise a fatty alcohol in order to provide a thickening effect. Similar to fatty acids, any fatty alcohol that is compatible with other components of the formulation and capable of being topically applied may be used. A fatty alcohol specifically trialled by the inventor is cetyl alcohol, which is used in the cosmetic industry as an opacifier in shampoos, or as an emollient, emulsifier or thickening agent in the manufacture of skin creams and lotions. Combinations of two or more fatty alcohols may be used, where appropriate.

In some embodiments, for example, the formulation may further comprise Vitamin E and/or a component thereof. Vitamin E is an antioxidant and is an important ingredient in many cosmetic products and is believed to protect the skin from various deleterious effects due to solar radiation. Vitamin E is a mixture including the stereoisomers gamma-tocopherol and alpha-tocopherol, which can be separately obtained and used in the formulation either in addition to, or in place of, the Vitamin E.

In a more specific aspect, the present invention provides topical formulations for treating psoriasis, comprising:
ground Triticum aestivum and/or ground Triticum vulgare;
Triticum aestivum germ extract and/or Triticum vulgare germ extract;
ground Hordeum vulgare;
Hordeum vulgare extract;
Cocos nucifera oil;
egg albumen;
egg vitellus;
oleic acid;
linoleic acid;
palmitic acid;
cetyl alcohol;
Cocos nucifera water;
Vitamin E;
gamma-tocopherol; and
alpha-tocopherol.

In even more specific embodiments of the present invention, the formulation comprises:
between about 2 and 10 wt % (e.g. 3.7 wt %) Triticum aestivum germ extract and/or between about 2 and 10 wt % (e.g. 3.7 wt %) Triticum vulgare germ extract;
between about 2 and 10 wt % (e.g. 3.7 wt %) ground Triticum aestivum and/or between about 2 and 10 wt % (e.g. 3.7 wt %) ground Triticum vulgare;
between about 2 and 10 wt % (e.g. 3.7 wt %) ground Hordeum vulgare;
between about 2 and 10 wt % (e.g. 3.7 wt %) Hordeum vulgare extract;
between about 5 and 30 wt % (e.g. 18%) olive oil;
between about 10 and 30 wt % (e.g. 20 wt %) Cocos nucifera oil;
between about 5 and 30 wt % (e.g. 11 wt %) egg albumen;
between about 5 and 30 wt % (e.g. 11 wt %) egg vitellus;
between about 0.5 and 3 wt % (e.g. 1 wt %) oleic acid;
between about 0.5 and 3 wt % (e.g. 1 wt %) linoleic acid;
between about 0.5 and 3 wt % (e.g. 1 wt %) palmitic acid;
between about 2 and 10 wt % (e.g. 5 wt %) cetyl alcohol;
between about 10 and 30 wt % (e.g. 10 wt %) Cocos nucifera water;
between about 10 and 30 wt % (e.g. 10 wt %) distilled water;
between about 0.5 and 3 wt % (e.g. 1 wt %) Vitamin E;
between about 0.5 and 3 wt % (e.g. 1 wt %) gamma-tocopherol; and
between about 0.5 and 3 wt % (e.g. 1 wt %) alpha-tocopherol.

In a preferred embodiment of the present invention, the formulation may comprise the ingredients in the proportions set out in the following Table 2.

TABLE 2
Composition of Formulation 2

| | | Preferred |
|---|---|---|
| | | amount |
| Ingredient | Function | (wt %) |
| triticum aestivum | Assists by nourishing the skin and | 3.7% |
| germ extract | preventing sun damage | |
| triticum vulgare | Assists by nourishing the skin and | 3.7% |
| germ extract | preventing sun damage | |
| Ground triticum | Increases the elasticity of the skin | 3.7% |
| aestivum | | |
| Ground triticum | Increases the elasticity of the skin | 3.7% |
| vulgare | | |
| Ground hordeum | Increases the elasticity of the skin | 3.7% |
| vulgare | | |
| hordeum vulgare | Increases the elasticity of the skin | 3.7% |
| extract | | |
| cocos nucifera oil | Highly moisturising for the skin | 20% |
| | and prevents the body from | |
| | aging and cell damage. | |
| egg albumen | Emulsifies the ingredients to | 11% |
| egg vitellus | combine. | |
| | Moisturises skin and improves | |
| | skin texture | |
| oleic acid | Anti-inflammatory properties | 1% |
| linoleic acid | Anti-inflammatory properties | 1% |
| palmitic acid | Anti-inflammatory properties | 1% |
| cetyl alcohol | Thickening agent | 5% |
| vitamin E | Helps maintain skin health | 1% |
| gamma-tocopherol | Helps maintain skin health | 1% |
| alpha-tocopherol | Helps maintain skin health | 1% |
| cocos nucifera water | To hydrate the skin | 10% |
| Distilled water | To hydrate the skin | qs |

The present invention also provides a method for treating psoriasis, which comprises applying the topical formulation of the invention to an affected area of a patient.

Any suitable treatment regimen may be applied, with the efficacy of the regimen being readily ascertainable simply by visually inspecting the patient's psoriasis post-treatment. Application would be as for any other topical formulation, with a sufficient quantity of the formulation being applied to the affected area on the patient's skin and spread over and rubbed in in a conventional manner.

Typically, the applied topical formulation would be allowed to remain on the patient for an extended period of time, in order for maximum effect. Allowing such an extended period of contact may necessitate covering the affected area (e.g. with a bandage or other covering such as a shower cap), so that the formulation isn't wiped off the patient's skin. Alternatively, however, a sufficient treatment may occur provided that the patient does not wash the affected area for the treatment period. In the inventor's experience, a shower is sufficient to remove any residual formulation on the patient.

The applied topical formulation may, for example, be allowed to remain on the patient for more than 1, 2, 3, 4, 5, 6, 7, 8, 9 or 10 hours. The applied topical formulation may, for example, be allowed to remain on the patient overnight.

The topical formulation may be re-applied as often and for as long as necessary in order to treat the psoriasis. The formulation may, for example, be applied on a daily basis until treatment is complete. The inventor has witnessed an overnight successful treatment, but notes that treatment may need to be continued for 1, 2, 3, 4, 5 or even more days in some circumstances.

The topical formulation may also be re-applied on a regular basis in order for the patient to remain substantially psoriasis free. Depending on factors such as the severity and extent of the patient's psoriasis, the formulation may be applied on a weekly, fortnightly or monthly basis.

As noted above, the present invention also provides for the use of topical formulations of the invention for treating psoriasis.

A method for producing a formulation in accordance with an embodiment of the present invention (Formulation 2, described above) will now be described.
Step 1. Prepare a double boiler on the heat.
Step 2. Pour the distilled water, Cocos nucifera water, Triticum aestivum germ extract, Triticum vulgare germ extract, ground Triticum aestivum, ground Triticum vulgare, ground Hordeum vulgare and Hordeum vulgare extract into the double boiler and begin to whisk the mixture slowly.
Step 3. Pour in the egg albumen and vitellus and whisk to combine all of the substances.
Step 4. As soon as the mixture is well combined, begin to add the oleic acid, linoleic acid, palmitic acid, and Cocos nucifera oil to the double boiler and continue to mix.
Step 5. In a separate double boiler begin to add in the cetyl alcohol and mix until it melts.
Step 6. Pour the melted cetyl alcohol into the mixture and whisk to combine the ingredients.
Step 7. Take the mixture bowl off of the heat and continue to whisk with a hand mixer for 4-5 minutes.
Step 8. Add in vitamin E, gamma-tocopherol and alpha-tocopherol.
Step 9. Pour the liquid into a container, cover it and store it in a fridge for it to set completely.
Step 10. After the liquid is set, the formulation is in the form of a cream that is ready for use.

### Examples

A formulation having the composition set out above and manufactured using the method set out above was topically applied to four children affected by psoriasis in the manner described below.

About 100 g of the formulation was applied to the dry scalp of the patient, which was exhibiting clear symptoms of psoriasis. The whole scalp was covered with the formulation, and not only the visually affected areas. The scalp was then covered with a shower cap, and the formulation left on the scalp overnight, or at least for an extended period of hours (e.g. 6-8 hours, the longer the better). In the morning the patient had a regular shower and combed their hair after the shower to remove any flakes. A major difference in the severity of the psoriasis could be seen at this point, indeed one of the patient's scalps was completely clear after only one treatment (the inventor notes that treatment times may vary depending on the severity of the psoriasis and from one person to another, and that it might therefore require a few applications for the psoriasis to clear completely for some individuals).

This treatment was applied once fortnightly until all signs of the psoriasis had completely cleared, and then performed once monthly for maintenance.

In other trials, topical formulations having in the composition set out above were applied to the scalps of three further patients (three adults; 2 males and a female). All were psoriasis sufferers and showed symptoms of scaling, irritation, itchiness, inflammation and redness. 80-120 grams of the formulation was used, depending on the individual's hair length. The formulation was applied on the scalp, and then the scalp was covered with a non absorbable head cap or plastic wrap and left for a period of at least 6-8 hours. (the longer the better).

In the morning, the patients showered as usual, and then combed their hair to remove any flakes. All patients showed a dramatic improvement in results after the first treatment, having reduced scaling, irritation, itchiness, inflammation and redness. One of the patients had a mild case of scalp psoriasis and their scalp was cleared of psoriasis after only one treatment. The other two patients had more severe cases (one having psoriasis on her scalp, ears and forehead), but were healed by at least 40% after the first treatment and were psoriasis free following the second treatment. Re-application of the formulation after a period of 2-4 weeks has kept these patients substantially psoriasis free.

In another trial, the formulation was topically applied to the scalps of twin boys who were suffering from mild to moderate psoriasis using the protocol described above. The psoriasis cleared up after only one application.

In another trial, the formulation was topically applied twice daily to the back and legs of a 37 yo man suffering from relatively severe eczema. No coverings were used in the treatment process, with the formulation being used in a manner more like that of a topical cream. The patient's eczema healed after 1-2 weeks, although he gained soothing relief after every application.

In summary, the invention described herein relates to formulations and methods for treating psoriasis and other skin conditions such as eczema. It will be appreciated from the disclosure set out above that the present invention provides a number of new and useful results.

For example, specific embodiments of the present invention may provide one or more of the following advantages:
an effective treatment for psoriasis that does not contain steroids or other ingredients which are associated with adverse side effects;
an effective treatment for psoriasis that includes primarily what are considered to be “Natural” ingredients, and therefore more likely to be attractive to some customers; and
a psoriasis treatment that is suitable for children.

It will be understood to persons skilled in the art of the invention that many modifications may be made without departing from the spirit and scope of the invention. All such modifications are intended to fall within the scope of the following claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A topical formulation for treating psoriasis, the formulation comprising:
ground wheat and a wheat extract;
ground barley and a barley extract;
an oil; and
egg or a fraction thereof.

2. The formulation of claim 1, wherein the ground wheat comprises wheat flour.

3. The formulation of claim 1, wherein the ground wheat comprises ground Triticum aestivum.

4. The formulation of claim 1, wherein the ground wheat comprises ground Triticum vulgare.

5. The formulation of claim 1, wherein the wheat extract comprises Triticum aestivum germ extract.

6. The formulation of claim 1, wherein the wheat extract comprises Triticum vulgare germ extract.

7. The formulation of claim 1, wherein the ground barley is obtained from one or more of the following: pearl barley, malted barley and hulled barley.

8. The formulation of claim 1, wherein the oil comprises olive oil.

9. The formulation of claim 1, wherein the oil comprises Cocos nucifera oil.

10. The formulation of claim 1, wherein the egg or a fraction thereof is provided in the form of any one or more of the following: whole egg, egg albumen, egg vitellus and extracted therapeutically active fractions of the egg.

11. The formulation of claim 1, wherein the formulation contains between about 2 wt % and 10 wt % of each of the ground wheat and wheat extract.

12. The formulation of claim 1, wherein the formulation contains between about 2 wt % and 10 wt % of each of the ground barley and barley extract.

13. The formulation of claim 1, wherein the formulation contains between about 10 wt % and 30 wt % of the oil.

14. The formulation of claim 1, wherein the formulation contains between about 5 wt % and 30 wt % of the egg or a fraction thereof.

15. The formulation of claim 1, further comprising a fatty acid and/or a fatty alcohol.

16. The formulation of claim 15, wherein the fatty acid is selected from one or more of the following: oleic acid, linoleic acid and palmitic acid.

17. (canceled)

18. The formulation of claim 15, wherein the fatty alcohol is cetyl alcohol.

19. The formulation of claim 1, further comprising Vitamin E or a component thereof.

20. A topical formulation for treating psoriasis, comprising:
ground Triticum aestivum and ground Triticum vulgare;
Triticum aestivum germ extract and Triticum vulgare germ extract;
ground Hordeum vulgare;
Hordeum vulgare extract;
Cocos nucifera oil;
egg albumen;
egg vitellus;
oleic acid;
linoleic acid;
palmitic acid;
cetyl alcohol;
Cocos nucifera water;
Vitamin E;
gamma-tocopherol; and
alpha-tocopherol.

21. A method for treating psoriasis, the method comprising: applying the topical formulation of claim 1 to an affected area of a patient.

22. (canceled)

23. (canceled)

24. (canceled)

25. (canceled)

26. (canceled)

27. (canceled)

28. (canceled)
